(19) United States
(12) Patent Application Publication
(10) Pub. No.: US 2008/0138409 A1
(43) Pub. Date: Jun. 12, 2008

(54) OLANZAPINE PHARMACEUTICAL COMPOSITION

(76) Inventor: Niels J. Osinga, Nijmegen (NL)

Correspondence Address: SYNTHON IP INC, 7130 HERITAGE VILLAGE PLAZA, STE 202, GAINESVILLE, VA, 20155, US

(21) Appl. No.: 11/863,795

(22) Filed: Sep. 28, 2007

Related U.S. Application Data
(63) Continuation of application No. 60/827,607, filed on Sep. 29, 2006.

(51) Int. Cl. A61K 9/20; A61K 31/5513

(52) U.S. Cl. 424/465; 514/220

(57) ABSTRACT

An olanzapine pharmaceutical composition such as a tablet is made using anhydrous lactose as an excipient.

The present application claims the benefit of priority under 35 U.S.C. § 119(e) from U.S. provisional application Ser. No. 60/827,607, filed Sep. 29, 2006, the entire contents of which are incorporated herein by reference.

### BACKGROUND OF THE INVENTION

The present invention relates to a solid pharmaceutical composition comprising olanzapine as the active ingredient.

Olanzapine is represented by the structural formula (1)

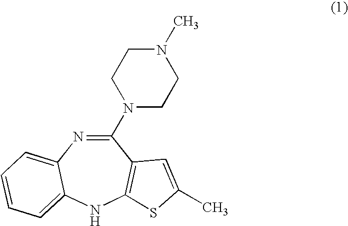

and is a pharmaceutically active compound. In medical treatments, it is useful as an antipsychotic agent, particularly for the treatment of schizophrenia. The marketed final forms include coated tablets and quick dissolvable tablets. The single tablet comprises from 2.5 to 20 mg of olanzapine.

While olanzapine may form acid addition salts, in the present commercially available final forms the active substance is marketed as a free base. It is a yellow crystalline solid that is insoluble in water (solubility at pH 6.8 is about 0.02 mg/ml).

Olanzapine and pharmaceutically acceptable salts have been suggested in EP 454436 and corresponding U.S. Pat. No. 5,229,382. Later, it became known that olanzapine (base) may exist in various crystalline modifications, including some hydrated/solvated forms, that are stable at ambient conditions (For example, see EP 733635/U.S. Pat. No. 5,736,541, WO 98-11893, EP 831098, U.S. Pat. No. 6,348,458 (WO 01/47933), WO 02/18390, WO 03/091260, WO 03/097650, WO 03/101997, WO 04/006933, US Appl. Publication No. 2002-0086993, and Reutzel-Edens et al. (Crystal Growth and Design, 2003, vol. 3, No. 6, 897-907)).

The term “Form I olanzapine” was later designated in EP 733635 to the anhydrous olanzapine product that was stated to be obtainable according to the process of U.S. Pat. No. 5,229,382.

As the system used for numbering the known olanzapine forms is sometimes confusing in the prior art disclosures (for instance, the EP 828494 calls as olanzapine Form I a product that is identical with olanzapine Form II of the above definition), the “Form I” of olanzapine as used herein is defined as the solid state form of anhydrous olanzapine base which is characterized by a typical peak on the X-ray powder diffraction spectrum of d-value of about 9.95 A. The full diffraction pattern of the Form I has been disclosed in EP 733635. The “Form II” of olanzapine as used herein has the same definition as used in EP 733635/U.S. Pat. No. 5,736,541, namely it is characterized by a typical X-ray powder diffraction peak of d-value of about 10.26 A.

In EP 0454436B1 it is reported that pharmaceutical compositions of olanzapine can be prepared by using conventional techniques. The active ingredient can be mixed with a carrier such as lactose; dextrose, sucrose, sorbitol, mannitol, starches, gum acacia, calcium phosphate, alginates, tragacanth, gelatin, syrup, methyl cellulose, methyl and propyl-hydroxybenzoate, talc, magnesium stearate or mineral oil. In a specific example there is given a formulation containing magnesium stearate, microcrystalline cellulose, povidone and starch. Depending on the method of administration, the compositions may be formulated as tablets, capsules, injection solutions for parenteral use, suspensions or elixirs for oral use, or suppositories.

EP 0733367 B1 relates to a stable solid oral formulation comprising olanzapine intimately mixed with a bulking agent, a binder, a disintegrant, a dry binder and a lubricant, whereupon such solid oral formulation is coated with a polymer. The coating with certain polymers is said to provide uniformity and physical stability and to effectively prevent the undesired discoloration phenomenon in the formulation. Ambient conditions, elevated temperatures and moist environment aggravate the problem of discoloration. The process for the preparation of the formulation comprises the steps of wet granulation, drying, blending with additional excipients and compression. The obtained cores are first sub-coated with HPMC and subsequently coated with a coating suspension. In the description it is pointed out that olanzapine may form an undesired crystal form in the presence of certain solvents and excipients, therefore it is desired to prepare the formulation using a method which does not require dissolving of the olanzapine substance. They believe that a dry blend direct compression process or a dry granulation process for preparing solid oral formulations create a greater chance for poor dose uniformity to occur. In the light of the potent nature of olanzapine, consistent dose uniformity is imperative. Therefore, they used high-shear aqueous wet granulation with fluid bed drying as the most effective method for preparing pharmaceutically elegant and stable oral olanzapine formulations. Though the presence of solvents can cause undesirable conversions they could not avoid the use of wet granulation.

EP 0830858 A1 relates to a formulation containing a coated active ingredient. The coating provides a uniform physical stability and effectively prevents the undesired discoloration phenomenon in the formulation. They stated that olanzapine undergoes undesirable discoloration when contacted with certain excipients including powder blends.

WO 2004/035027 provided a formulation with high stability without any undesired discoloration or poor dose uniformity. The formulation comprises a homogeneous mixture of (a) olanzapine or a pharmaceutically acceptable salt thereof as an active ingredient, (b) a monosaccharide and/or oligosaccharide, (c) a polysaccharide and, optionally, further ingredients. According to a preferred embodiment, the tablets are prepared by direct compression. It was found out that the discoloration phenomenon is probably caused by the formation of olanzapine hydrates. In order to prevent the formation of hydrates, the process for the manufacture of the pharmaceutical formulation should be performed without using solvents. It was also found out that stable pharmaceutical formulations comprising olanzapine can be prepared by a simple direct compression process if olanzapine or a pharmaceutically acceptable salt thereof is first homogeneously mixed with certain excipients and then subjected to direct compression. The direct compression is performed in the absence of any solvent. The specific excipients used allow the production of stable olanzapine formulations without any need for a coating or wet granulation.

WO 2003/086361 relates to rapidly dispersing solid oral compositions comprising olanzapine. Examples 7-12 describe compositions with olanzapine using wet granulation methodology. Example 13 gives a tablet composition with olanzapine using direct compression technique. The description mentions that alternatively the tablets can be obtained using direct compression technique.

WO 2005/0009407 disclosed formulations stable to discoloration comprising lactose and/or mannitol-coated OPN particles.

It is desirable to provide a stable pharmaceutical composition comprising olanzapine or a salt thereof, that is simple and relatively easy to manufacture. Preferably a tablet that can be made using conventional/typical direct compression techniques and that has good content uniformity, good chemical stability and/or a low tendency to a colour change, would be beneficial.

### SUMMARY OF THE INVENTION

The present invention relates to a solid state pharmaceutical composition comprising olanzapine, or a pharmaceutically acceptable salt thereof, and anhydrous lactose. Typically the composition is in the form of a tablet for oral administration, which comprises an effective amount of olanzapine or a pharmaceutically acceptable salt thereof, anhydrous lactose, and a disintegrant. The anhydrous lactose typically comprises 40-95% of the tablet by weight. The tablet can contain other excipients such as a lubricant/glidant. The tablet can be made simply by a direct compression method. Thus, a suitable method may comprise blending the olanzapine or its salt, anhydrous lactose, disintegrant, and optionally other excipients in one or more steps to form a powder blend and then compressing the powder blend to form the tablets. Surprisingly, the use of anhydrous lactose allows for good content uniformity, and good stability to both discoloration and chemical breakdown.

### DETAILED DESCRIPTION

Within the present invention, the word “olanzapine” covers only the anhydrate form of olanzapine base, not a hydrate nor a solvate thereof, unless otherwise specifically indicated.

During the thorough research on tablettable pharmaceutical compositions comprising olanzapine as the active ingredient, and particularly the solid state Form I thereof, it was found out that many compositions suffer from a progressive formation of an undesirable impurity, which has been identified as a lactam compound 2-methyl-5,10-dihydro-4H-thieno[2,3-b][1,5]benzodiazepin-4-one of the formula (II)

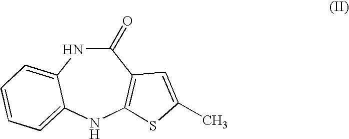

In attempts to reduce the amounts of the lactam impurity formed during prolonged storage of olanzapine compositions, it was found out that its formation may be suppressed in compositions that contain anhydrous lactose.

Furthermore, it was found out that compositions containing anhydrous lactose have a low tendency to color change, which is an important aspect addressed in many prior art disclosures. Indeed, in tablet form, for example, compositions according to the present invention can be stable to color change even without the use of pre-coated olanzapine particles as taught in WO 2005/0009407.

Additionally, it was found that the composition can be formed with very good content uniformity even when no liquid has been used within the homogenization process. Thus anhydrous lactose can enable the use of direct compression techniques in forming a good quality olanzapine tablet.

Two basic components of the composition of the present invention are anhydrous lactose and solid state olanzapine or a salt thereof.

Lactoses are well known pharmaceutical excipients for use in making tablets and capsules. Typically the excipient “lactose” refers to lactose monohydrate which has a theoretical water of content of 5% and in practice contains 4.5-5.5% by weight. However, anhydrous lactose is also known to be used as an excipient and various grades thereof are also available. For purposes of the present invention, “anhydrous lactose” means a lactose composition/substance having a water content of less than 1% by weight, preferably 0.5% or less, and more preferably 0.3% or less. For clarity, as used in the present application, the water content amounts are referring to the amounts as determined by Karl-Fischer analysis.

The chemical structure of lactose consists of two sugar moieties, which can form the disaccharide in two different conformations; namely α-lactose and β-lactose. The anhydrous lactose used in the present invention generally contains at least 50%, and typically 60-90% of anhydrous β-lactose. Commercially available anhydrous β-lactose grades typically contain as little as 70% anhydrous β-lactose, with the remainder comprising anhydrous α-lactose. Some grades contain at least 80%, e.g. 80-90%, of anhydrous β-lactose and the remaining being anhydrous α-lactose. In general, higher anhydrous β-lactose content is preferred for use in the present invention, typically in the range of 75-85%.

The anhydrous lactose is typically crystalline or substantially crystalline in nature. The anhydrous lactose is generally a free flowing powder in order to be readily combined with olanzapine via direct compression techniques. The bulk density of the lactose particle population is typically between 0.60-0.70 g/cm³ and the specific surface area between 0.3 and 0.5 m²/g, though these properties are not required. The above parameters can be determined by methods known in the art.

The particle population making up the powder typically has an average particle size in the range of 20 to 250 microns, more typically 50-175 microns. In some embodiments, it is desirable that the population include relatively large particles. For instance, a d₅₀ greater than 100 microns, typically between 100 and 250 microns (d₅₀ of a mass distribution determined by sieving method) can be useful. Generally such populations have a d₃₅ in the range of 80-160 microns and a d₇₅ of at least 175 microns, typically 175-275 microns (all based on mass distribution determined by a sieving method). Preferred populations can also be described as follows:

| | | Weight of particles | |
|---|---|---|---|
| | | within the particle | |
| Particle size range | | size range | Typical values |
| <45 | microns | 20% or less | 5%, 15% |
| <150 | microns | 30-65% | 35%, 50% |
| <250 | microns | At least 70% | 75%, 85% |

Commercially available anhydrous lactose grades suitable for use in the present invention are sold under the brand name Pharmatose® (DMV International). In particular, Pharmatose DCL 21 and Pharmatose DCL 22 are preferred anhydrous lactose grades. The typical values recited in the above table correspond to DCL 21 (15, 50, 85) and DCL 22 (5, 35, 75) as reported in the DMV International brochure describing its Pharmatose brand lactose.

The second component of the composition is olanzapine or a pharmaceutically acceptable salt thereof, in solid state. Any known pharmaceutically acceptable acid can be used to make a salt of olanzapine and such salts are suitable as long as they are solids. Specific examples of suitable acids include hydrochloric, hydrobromic, acetic, benzoic, methane sulfonic, benzene sulfonic, sulfuric, maleic, fumaric, tartatic, lactic, phosphoric, citric, and malic acids. Typically the olanzapine component is either the free base or olanzapine benzoate. Normally the olanzapine component is crystalline.

As discussed above, crystalline olanzapine may exist in various polymorphic forms as well as in an amorphous, i.e. noncrystalline, form. The invention is not limited to a specific polymorph or form of the solid state olanzapine component and any of the above mentioned forms of olanzapine are contemplated for use in the present invention. However, it has been observed that the present invention can be particularly useful in formulating compositions comprising crystalline olanzapine Form I. Although Form I is thermodynamically somewhat less stable than Form II, it was surprisingly found that compositions of the present invention comprising the Form I still show a low tendency of polymorphic/pseudopolymorphic changes of the crystal structure of olanzapine and thus are not prone to excessive discoloration. Additionally, free base olanzapine such as Form I is generally preferred over an olanzapine salt for stability and/or handling reasons.

The olanzapine, particularly the Form I olanzapine, is not specifically limited in particle size. Indeed, larger particles can be used which can be advantageous in that the need to mill or sieve in order to reduce the native particle size population can be avoided. Not only can this save a unit operation step, but also it can avoid applying a potential polymorphic/pseudopolymorphic altering stress. Thus, olanzapine such as crystalline Form I olanzapine, having a d₅₀ (of a volume distribution) in the range of 90-120 micron can be successfully combined with anhydrous lactose, preferably having a similar large particle size distribution such as DCL 21 or DCL 22, by normal blending procedures.

The olanzapine particles are not required to be pre-coated with a discoloration preventing coating or layer as taught in EP 0 830 858, and preferably are not coated as described in EP 0 830 858 before their use in making the composition. In general the olanzapine particles have no coating of any kind as such requires an extra step.

The compositions of the present invention can take the form of powder blends, capsules, tablets, etc. Typically the amount of olanzapine is 1 to 10%, and more typically 2 to 7% and frequently 3 to 6% or 4 to 5%, of the composition, by weight, and the amount of anhydrous lactose is generally at least 40%, and more typically at least 50% and frequently 70 to 95% (such as 90 to 95%) of the composition, by weight.

One embodiment of the composition of the present invention is a tablet for oral administration. The oral pharmaceutical tablet comprises an effective amount of olanzapine or a pharmaceutically acceptable salt thereof, anhydrous lactose, and a disintegrant. The amount of olanzapine component is typically 1-10% by weight and frequently 4-5%, and in absolute terms is generally 1-50 mg, such as 5, 10, 15, or 20 mg, expressed in terms of the amount of olanzapine base. The anhydrous lactose comprises 40-95% of the tablet by weight. Generally higher amounts of anhydrous lactose are preferred such as 80-95% and even 90-95%, and in a preferred embodiment anhydrous lactose is the only diluent used in the tablet. However, it is possible to replace some of the anhydrous lactose with other diluents or filler-binders, such as mannitol, sucrose, sorbitol, maltodextrin, etc., and applying the general rule of thumb that up to half of a diluent/binder can be replaced with another, it is believed that as little as 40-50% of anhydrous lactose would provide beneficial results. Nonetheless, greater than 50% anhydrous lactose is preferred, especially at least 70% and more typically 80-95% as mentioned above.

The disintegrant can, in essence, be any solid-state disintegrant. Of course, it should exhibit sufficient compressibility and good flow properties so as to be practical in making tablets. Typical disintegrants include sodium starch glycolate and crosspovidone, which is a crosslinked poly (N-vinyl-2 pyrrolidone). Crosspovidone is generally preferred and can be commercially obtained in pharmaceutical grade, e.g., Polyplasdone XL. The amount of the disintegrant is usually minor, generally from 1 to 10 weight %.

Additionally the oral tablet will frequently contain one or more compounds from the lubricant/glidant family to improve the flow properties within the tabletting process and/or minimize the stickiness to tablet punches. Suitable lubricants/glidants include magnesium stearate, sodium stearyl fumarate, calcium stearate, glyceryl behenate, talc, and/or colloidal silicon dioxide. Often two or more of such compounds are used in differing amounts. The total amount of the lubricant/glidant is generally minor and frequently less than 10%, such as 0.25-5%, by weight.

The tablet can contain other auxiliary excipients such as colorants, flavorants, etc. It is desirable that the auxiliary components are anhydrous, or have a low water content. Indeed, in general the total water content of the tablet is normally less than 2%, preferably less than 1%, such as 0.8% or 0.5% (Karl Fischer water content analysis), as it is believed that water is a source of the discoloration problems. The mass of the tablet is typically from 75 to 500 mg, with tablets of 100 and 400 mg being specifically contemplated. The tablets normally exhibit a hardness from about 30 to about 130 N.

The tablets can be formed by any conventional methods, but preferably are made by a dry process, that is one without the use of a liquid such as water to aide in the mixing/blending operation. Not only is wet granulation preferably avoided, but even dry granulation (e.g., by roller compaction) is normally not necessary to achieving a quality pharmaceutical oral tablet.

Accordingly, the tablets of the invention are preferably made by a process that comprises blending olanzapine or a pharmaceutically acceptable salt thereof, anhydrous lactose, and a disintegrant in one or more mixing steps to form a powder blend, and then compressing the powder blend to form tablets. The blending or mixing of the components need not blend all of the components and/or the full amount of a component in each mixing step, e.g. the blending of only a portion of the total amount of a component and/or omitting a component until the final mixing step, etc., are all contemplated. Sieving may optionally be performed before and/or after one or more mixing steps.

The resulting powder blend is compressed into tablets by conventional techniques. Generally this is achieved by supplying a small portion of the powder blend to a dye and compressing the blend. The shape and size of the dye and hence the produced tablet are not particularly limited and include round and oval with flat or biconvex faces, but is not limited thereto. A typical compression force used to make the tablets is from 4 to 20 kN.

The tablets can be coated if desired. However, it is preferred that the tablets are not coated with a discoloration-preventing layer as described in EP 0 733 367. And typically the tablets are uncoated.

The use of anhydrous lactose as described above, especially in higher amounts, can provide a tablet with good content uniformity and without significant or undesirable segregation. This is achieved without the use of water or other liquid in the blending/mixing step. And because the use of water or other solvent can be avoided, the risk of conversion of the olanzapine component, e.g. to a hydrate, a solvate or a different crystalline form, etc., is also lessened/avoided. The tablets exhibit good stability against color changes, even when the tablet does not possess a discoloration-preventing coating, nor do the olanzapine particles contain such a protective coating. The tablets also show good chemical stability such as in the reduction of the formation of the lactam of formula II.

The tablets may be used in the treatment of olanzapine-treatable diseases in dosages and regimens similar to the marketed olanzapine tablets. Generally the tablets are useful in the treatment of schizophrenia. The invention will now be described by way of the following non-limiting examples.

### EXAMPLE 1

TABLE 1

| | Formulation I | | | Formulation II | | |
|---|---|---|---|---|---|---|
| Olanzapine form I | 5.0 | mg | 2.5% | 20.0 | mg | 6.25% |
| Pharmatose DCL22 | 186.5 | mg | 93.25% | 286.4 | mg | 89.5% |
| (anhydrous lactose) | | | | | | |
| Aerosil 200 VV | 0.5 | mg | 0.25% | 0.8 | mg | 0.25% |
| (colloidal silicon dioxide) | | | | | | |
| Polyplasdone XL | 6.0 | mg | 3.0% | 9.6 | mg | 3% |
| (crosspovidone) | | | | | | |
| Magnesium stearate | 2.0 | mg | 1.0% | 3.2 | mg | 1% |
| Total | 200.0 | mg | 100% | 320.0 | mg | 100% |

The above formulations can be produced by:

(A) Sieving of olanzapine, aerosil and part of lactose (1:3 ratio) through a 355 um sieve, and blending together in a Turbula mixer for 20 min at 46 RPM.
(B) Sieving the remaining lactose and crosspovidone through 600 um sieve, adding the blend of (A), and blending all together in the Turbula mixer for 20 min at 46 RPM.
(C) Sieving the magnesium stearate through 600 um sieve, adding it to the blend of (B), and blending in Turbula mixer for 3 min at 46 RPM.
(D) Compressing the blend of (C) into tablets on a Korsch EK-0 excenterpress.

TABLE 2
Properties of olanzapine base form I formulations

| | Formulation I | Formulation II |
|---|---|---|
| Properties | 5 mg olanzapine | 20 mg olanzapine |
| Flowability (g/sec) | | |
| Ø 2.5 cm funnel | 59 | 42 |
| Ø 1.5 cm funnel | 16 | 11 |
| Ø 1.0 cm funnel | 6 | 4 |
| Bulk density (g/ml) | 0.632 | 0.645 |
| Tapped density (g/ml) | 0.779 | 0.765 |
| LOD (%; 105° C.) | 0.5 | — |
| pH 20% slurry | — | 8.70 |
| Weight variation | 203.4 ± 0.27% | 322.2 ± 0.12% |
| (mg; % RSD) | | |
| Disintegration (min) | <2 | <2 |
| Hardness (N) | 39 | 54 |
| Friability (%) | 0.10 | — |

### Comparative Example 1

| | Formulation III | | | Formulation IV | | |
|---|---|---|---|---|---|---|
| Olanzapine form I | 20.0 | mg | 6.25% | 20.0 | mg | 6.25% |
| Ludipress | 296.8 | mg | 92.75% | | | |
| (lactose monohydrate/ | | | | | | |
| povidone/crosspovidone) | | | | | | |
| Pharmatose DCL 14 | | | | 222.4 | mg | 69.5% |
| (lactose monohydrate) | | | | | | |
| Lactochem crystals | | | | 64.0 | mg | 20.0% |
| (lactose monohydrate) | | | | | | |
| Aerosil 200 VV | — | | — | 0.8 | mg | 0.25% |
| (colloidal silicon dioxide) | | | | | | |
| Polyplasdone XL | — | | — | 9.6 | mg | 3.0% |
| (crosspovidone) | | | | | | |
| Magnesium stearate | 3.2 | mg | 1.0% | 3.2 | mg | 1.0% |
| Total | 320.0 | mg | 100% | 320.0 | mg | 100% |

The formulations III and IV were prepared in a similar way as the formulations I and II. The formulations III and IV both suffered from segregation problems of the olanzapine which resulted in tablets with inhomogeneous colour; i.e., the yellow color olanzapine was clearly more concentrated around the edges which makes the tablet inelegant and unacceptable. In contrast, Formulations I and II produced pharmaceutically elegant tablets that did not suffer from segregation/content uniformity issues.

Each of the patents, patent applications, and journal articles mentioned above are incorporated herein in their entirety by reference. The invention having been described it will be obvious that the same may be varied in many ways and all such modifications are contemplated as being within the scope of the invention as defined by the following claims.

1. A solid state pharmaceutical composition comprising olanzapine or a pharmaceutically acceptable salt thereof and anhydrous lactose.

2. The composition according to claim 1, wherein said anhydrous lactose has a water content of 0.5% or less.

3. The composition according to claim 2, wherein said anhydrous lactose is at least 50% anhydrous β-lactose.

4. The composition according to claim 3, wherein said anhydrous lactose is 60-90% anhydrous β-lactose.

5. The composition according to claim 4, wherein said anhydrous lactose has an average particle size in the range of 50-175 microns

6. The composition according to claim 5, wherein said anhydrous lactose has a d₃₅ in the range of 80-160 microns and a d₇₅ of at least 175 microns.

7. The composition according to claim 6, wherein said anhydrous lactose has a d₇₅ of 175-275 microns.

8. An oral pharmaceutical tablet comprising an effective amount of olanzapine or a pharmaceutically acceptable salt thereof, anhydrous lactose, and a disintegrant, and wherein said anhydrous lactose comprises 40-95% of said tablet by weight.

9. The tablet according to claim 8, wherein said olanzapine is olanzapine free base.

10. The tablet according to claim 9, wherein said olanzapine is contained in an amount between 2.5 and 50 mg.

11. The tablet according to claim 10, wherein said olanzapine is contained in an amount of 1-10% of the tablet, by weight.

12. The tablet according to claim 10, wherein said anhydrous lactose comprises 70-95% of said tablet, by weight.

13. The tablet according to claim 8, wherein said anhydrous lactose has a water content of 0.5% or less.

14. The tablet according to claim 8, wherein said anhydrous lactose is at least 50% anhydrous. β-lactose.

15. The tablet according to claim 12, wherein said anhydrous lactose is 60-90% anhydrous β-lactose.

16. The tablet according to claim 8, wherein said disintegrant is a crosspovidone.

17. The tablet according to claim 8, which further comprises at least one lubricant/glidant selected from magnesium stearate, sodium stearyl fumarate, calcium stearate, glyceryl behenate, talc, and colloidal silicon dioxide.

18. The tablet according to claim 8, wherein said olanzapine is uncoated.

19. The tablet according to claim 8, which does not contain a discoloration-preventing coating.

20. A process for making the tablet according to claim 8, which comprises blending olanzapine or a pharmaceutically acceptable salt thereof, anhydrous lactose, and a disintegrant in one or more steps to form a powder blend, and compressing said powder blend to form said tablet.
